(19) United States
(12) Patent Application Publication
(10) Pub. No.: US 2008/0028835 A1
(43) Pub. Date: Feb. 7, 2008

(54) Apparatus of supplying and containing micro-plate, method of supplying and method of containing micro-plate and apparatus of processing micro-plate

(75) Inventor: Akira Higuchi, Fukuoka (JP)

Correspondence Address: PEARNE & GORDON LLP, 1801 EAST 9TH STREET, SUITE 1200, CLEVELAND, OH, 44114-3108, US

(73) Assignee: MATSUSHITA ELECTRIC INDUSTRIAL CO., LTD., Osaka (JP)

(21) Appl. No.: 10/598,510

(22) PCT Filed: Mar. 3, 2005
(86) PCT No.: PCT/JP05/04139

§ 371 Date: Sep. 1, 2006

(30) Foreign Application Priority Data
Mar. 5, 2004 (JP) 2004-061990

(51) Int. Cl. G01N 33/487

(52) U.S. Cl. 73/53.01

(57) ABSTRACT

It is an object of the invention to provide an apparatus of supplying a micro-plate excellent in operating performance and operability.

In a micro-plate supplying apparatus for separating micro-plates 10 mounted in a stacked state sheet by sheet to supply, a first stock portion 2, a second stock portion 3 for containing the micro-plates in the stacked state are arranged in series in an up and down direction by interposing a micro-plate take out and take in mechanism 4, the micro-plates 10 are moved from the first stock portion 2 to the second stock portion 3 while staying in the stacked state, the micro-plate at a uppermost stage of the plurality of micro-plates 10 is disposed at a micro-plate carry out level L2, and the micro-plate is taken out by a micro-plate carrying out mechanism to supply to a working apparatus 5. Thereby, the micro-plate is set in an excellent operation attitude and the micro-plate can stably be taken out.

### FIELD OF THE INVENTION

The present invention relates to an apparatus of supplying and containing a micro-plate for supplying and containing a micro-plate provided with a plurality of wells for containing a liquid as well as a method of supplying and a method of containing a micro-plate.

### BACKGROUND INFORMATION

In a drug creating screening field, a biotechnology or the like, a test of a biochemical reaction of a substance or the like is carried out, and a micro-plate is used as a vessel of containing a chemical solution for carrying out culture or biological reaction or a liquid of a specimen or the like. These tests are frequently carried out systematically by normally constituting an object by a number of samples and therefore, in a single test, a plurality of micro-plates constitute a processing object of dividedly injecting operation, composition analysis or the like.

Therefore, an exclusive processing apparatus for automatically carrying out such a processing is attached with an apparatus of supplying a micro-plate for stocking a plurality of micro-plates and supplying the micro-plates to the processing apparatus sheet by sheet (for example, Japanese Patent Publication No. 3260237). The reference example shows an example of supplying a micro-plate to a dividedly injecting apparatus for automatically carrying out dividedly injecting operation and the example is provided with two of stackers having a function of containing a plurality of micro-plates in a stacked state.

In supplying the micro-plate, the micro-plate constituting an object of test is taken out by being separated from a lower side of one stacker sheet by sheet and is delivered to the processing apparatus by a carrier. Further, the micro-plate finished with a processing is delivered to a lower side of other stacker by a carrier and is recovered into the stacker from a lower side. In the above-described reference example, the two stackers are made to be able to deal with either of a supply use of delivering the micro-plate from an opening provided at a lower end thereof and a recovery use of taking in the micro-plate from the opening.

Meanwhile, according to a test of a biochemical reaction or the like, in many cases, it is necessary to repeat a plurality of times of operation by constituting an object by the same micro-plate and in such a case, the micro-plate after finishing with a unit operation is temporarily recovered to the stacker in a stacked state and thereafter, taken out from the stacker and supplied to the processing apparatus. That is, the two stackers provided to the apparatus are alternately used for the supply use and the recovery use.

When a plurality of times of operation are executed by constituting an object by a plurality of micro-plates, generally, it is requested to maintain an order of processing respective micro-plates constant. However, in a system of using two stackers alternately for a supply use and a recovery use as in the above-described apparatus, when the micro-plate is taken out as it is from the stacker used for recovery, the micro-plate at a final order in proceeding operation is taken out as a micro-plate at an initial order in succeeding operation and a processing order thereof is reversed.

When the processing order is intended to be constant by avoiding such a drawback, there is needed restacking operation of realigning an order of stacking micro-plates in respective stackers. The restacking operation is complicated operation of taking out all the micro-plates from one stacker and setting the micro-plates to other stacker in a reverse order and such operation needs to execute for each unit operation. Therefore, in a drug creating screening which is carried out by constituting an object by a number of micro-plates, the restacking operation constitutes a factor of hampering an efficiency of test operation from being promoted.

### SUMMARY OF THE INVENTION

Hence, it is an object of the invention to provide an apparatus of supplying and containing a micro-plate as well as a method of supplying and a method of containing a micro-plate in the apparatus of supplying and containing a micro-plate in a test constituting an object by a plurality of micro-plates capable of promoting an efficiency of test operation without needing restacking operation.

According to the invention, there is provided an apparatus of supplying and containing a micro-plate characterized in comprising a first stock portion including a first support member for supporting a plurality of micro-plates in a stacked state from a lower side and a support release mechanism for changing the first support member into a state of not supporting the micro-plates, a second stock portion having a long vertical length arranged on a lower side of the first stock portion in series in an up and down direction, a second support member for supporting the plurality of micro-plates in the stacked state from a lower side at an inner portion of the second stock portion, a moving up and down mechanism for moving up and down the second support member, and micro-plate carrying means for carrying the micro-plate disposed at a micro-plate carry out level set at the second stock portion to outside and mounting the micro-plate to the second support member from the outside, further comprising a micro-plate supplying operation processing unit for making the support release mechanism and the moving up and down mechanism execute an operation of moving up the second support member and lifting the micro-plates in the stacked state supported by the first support member by the second support member to switch to mount on the second support member, thereafter, changing the first support member into a state of not supporting the micro-plates, thereafter, moving down the second support member and moving the plurality of micro-plates in the stacked state to the second stock portion, and disposing the micro-plate at a uppermost stage in the stacked state to the micropalte carry out level by controlling a height position of the second support member, and a micro-plate containing operation processing unit for making the moving up and down mechanism execute an operation of moving up the second support member mounted with the micro-plate delivered by the micro-plate carrying means to a height of capable of supporting the micro-plate by the first support member and thereafter, supporting the micro-plate by the first support member by moving down the second support member.

According to the invention, there is provided a method of supplying a micro-plate characterized in a method of supplying a micro-plate in an apparatus of supplying and containing a micro-plate comprising a first stock portion including a first support member for supporting a plurality of micro-plates in a stacked state from a lower side and a support release mechanism for changing the first support member into a state of not supporting the micro-plates, a second stock portion having a long vertical length arranged on a lower side of the first stock portion in series in an up and down direction, a second support member for supporting the plurality of micro-plates in the stacked state from a lower side at an inner portion of the second stock portion, a moving up and down mechanism for moving up and down mechanism for moving up and down the second support member, and micro-plate carrying means for carrying the micro-plate disposed at a micro-plate carry out level set at the second stock portion to outside and mounting the micro-plate to the second support member from the outside, the method comprising:

a step of moving up the second support member and lifting the micro-plates in the stacked state supported by the first support member by the second support member to switch to mount on the second support member;

a step of changing the first support member into a state of not supporting the micro-plates by operating the support release mechanism;

a step of moving down the second support member and moving the micro-plates in the stacked state which have been switched to mount on the second support member to the second stock portion;

a step of disposing the micro-plate at a uppermost stage in the stacked state to the micro-plate carry out level by controlling a height position of the second support member; and

a step of carrying out the micro-plate disposed at the micro-plate carry out level to the outside by the micro-plate carrying means.

According to the invention, there is provided a method of containing a micro-plate characterized in a method of containing a micro-plate in an apparatus of supplying and containing a micro-plate comprising a first stock portion including a first support member for supporting a plurality of micro-plates in a stacked state from a lower side and a support release mechanism for changing the first support member into a state of not supporting the micro-plates, a second stock portion having a long vertical length arranged on a lower side of the first stock portion in series in an up and down direction, a second support member for supporting the plurality of micro-plates in the stacked state from a lower side at an inner portion of the second stock portion, a moving up and down mechanism for moving up and down the second support member, and micro-plate carrying means for carrying the micro-plate disposed at a micro-plate carry out level of the second stock portion to outside and mounting the micro-plate to the second support member from the outside, said method comprising:

a step of mounting the micro-plate to the second support member by the micro-plate carrying means, and a step of moving down the second support member mounted with the micro-plate delivered by the micro-plate carrying means to a height of capable of supporting the micro-plate by the first support member and thereafter supporting the micro-plate by the first support member by moving down the second support member.

According to the invention, there is provided an apparatus of processing a micro-plate characterized in comprising a first apparatus of-supplying and containing a micro-plate capable of containing a plurality of micro-plates in a stacked state, a second apparatus of supplying and containing a micro-plate capable of containing a plurality of micro-plate in a stacked state, a working unit for executing a predetermined processing operation for the micro-plates, and a micro-plate carrying mechanism for carrying the micro-plates from the first apparatus of supplying and containing the micro-plate to the second apparatus of containing and supplying the micro-plate via the working unit, or carrying the micro-plates from the second apparatus of supplying and containing the micro-plate to the first apparatus of supplying and containing the micro-plate via the working unit;

wherein the first apparatus of supplying and containing the micro-plate and the second apparatus of supplying and containing the micro-plate each comprises a first stock portion including a first support member for supporting a plurality of micro-plates in a stacked state from a lower side and a support release mechanism for changing the first support member into a state of not supporting the micro-plates, a second stock portion having a long vertical length arranged on a lower side of the first stock portion in series in an up and down direction, a second support member for supporting the plurality of micro-plates in the stacked state from a lower side at an inner portion of the second stock portion, a moving up and down mechanism for moving up and down the second support member, and micro-plate carrying means for carrying the micro-plate disposed at a micro-plate carry out level set at the second stock portion to outside and mounting the micro-plate to the second support member from the outside, further comprising:

a micro-plate supplying operation processing unit for making the support release mechanism and the moving up and down mechanism execute an operation of moving up the second support member and lifting the micro-plates in the stacked state supported by the first support member by the second support member to switch to mount on the second support member, thereafter, changing the first support member into a state of not supporting the micro-plates, thereafter, moving down the second support member and moving the plurality of micro-plates in the stacked state to the second stock portion, and disposing the micro-plate at a uppermost stage in the stacked state to the micropalte carry out level by controlling a height position of the second support member; and

a micro-plate containing operation processing unit for making the moving up and down mechanism execute an operation of moving up the second support member mounted with the micro-plate delivered by the micro-plate carrying means to a height of capable of supporting the micro-plate by the first support member and thereafter, supporting the micro-plate by the first support member by moving down the second support member.

According to the invention, by adopting the constitution arranged with the first stock portion including the first support member capable of supporting and releasing support of the micro-plates in the stacked state and the second stock portion including the second support member supporting the micro-plates in the stacked state and capable of moving up and down in series in the up and down direction and including the micro-plate carrying means capable of receiving and delivering the micro-plate disposed at the micro-plate carry out level of the second stock portion to and from the outside, a micro-plate supplying operation of moving the micro-plates in the stacked state contained in the first stock portion to the second stock portion to supply to the outside successively from the micro-plate at the uppermost stage, and a micro-plate containing operation of containing the micro-plates delivered from the outside successively to the first stock portion are made to be able to execute by the same apparatus, a restacking operation of the micro-plates is not needed and efficiency of test operation can be promoted.

### BRIEF DESCRIPTION OF THE DRAWINGS

FIG. 1 is a front view of a working apparatus according to Embodiment 1 of the invention.

FIG. 2 is a side view of the working apparatus according to Embodiment 1 of the invention.

FIG. 3 illustrates explanatory views of total operation of the working apparatus according to Embodiment 1 of the invention.

FIG. 4 is a side view of an essential portion of a micro-plate supplying and containing apparatus according to Embodiment 1 of the invention.

FIG. 5 is a partial sectional view of the micro-plate supplying and containing apparatus according to Embodiment 1 of the invention.

FIG. 6 is a partial sectional view of the micro-plate supplying and containing apparatus according to Embodiment 1 of the invention.

FIG. 7 is a partial sectional view of the micro-plate supplying and containing apparatus according to Embodiment 1 of the invention.

FIG. 8 is a partial sectional view of the micro-plate supplying and containing apparatus according to Embodiment 1 of the invention.

FIG. 9 is a block diagram showing a constitution of a control system of the working apparatus according to Embodiment 1 of the invention.

FIG. 10 is an operation explanatory view of an operation of supplying a micro-plate in the micro-plate supplying and containing apparatus according to Embodiment 1 of the invention.

FIG. 11 is an operation explanatory view of the operation of supplying the micro-plate in the micro-plate supplying and containing apparatus according to Embodiment 1 of the invention.

FIG. 12 is an operation explanatory view of the operation of supplying the micro-plate in the micro-plate supplying and containing apparatus according to Embodiment 1 of the invention.

FIG. 13 is an operation explanatory view of the operation of supplying the micro-plate in the micro-plate supplying and containing apparatus according to Embodiment 1 of the invention.

FIG. 14 is an operation explanatory view of the operation of supplying the micro-plate in the micro-plate supplying and containing apparatus according to Embodiment 1 of the invention.

FIG. 15 is an operation explanatory view of the operation of supplying the micro-plate in the micro-plate supplying and containing apparatus according to Embodiment 1 of the invention.

FIG. 16 is an operation explanatory view of the operation of supplying the micro-plate in the micro-plate supplying and containing apparatus according to Embodiment 1 of the invention.

FIG. 17 is an operation explanatory view of the operation of supplying the micro-plate in the micro-plate supplying and containing apparatus according to Embodiment 1 of the invention.

FIG. 18 is an operation explanatory view of the operation of supplying the micro-plate in the micro-plate supplying and containing apparatus according to Embodiment 1 of the invention.

FIG. 19 is an operation explanatory view of operation of containing a micro-plate in the micro-plate supplying and containing apparatus according to Embodiment 1 of the invention.

FIG. 20 is an operation explanatory view of the operation of containing the micro-plate in the micro-plate supplying and containing apparatus according to Embodiment 1 of the invention.

FIG. 21 is an operation explanatory view of the operation of containing the micro-plate in the micro-plate supplying and containing apparatus according to Embodiment 1 of the invention.

FIG. 22 is an operation explanatory view of the operation of containing the micro-plate in the micro-plate supplying and containing apparatus according to Embodiment 1 of the invention.

FIG. 23 is an operation explanatory view of the operation of containing the micro-plate in the micro-plate supplying and containing apparatus according to Embodiment 1 of the invention.

FIG. 24 is an operation explanatory view of the operation of containing the micro-plate in the micro-plate supplying and containing apparatus according to Embodiment 1 of the invention.

FIG. 25 is a side view of an essential portion of a micro-plate supplying and containing apparatus according to Embodiment 2 of the invention.

FIG. 26 is an operation explanatory view of operation of supplying a micro-plate in the micro-plate supplying and containing apparatus according to Embodiment 2 of the invention.

FIG. 27 is an operation explanatory view of the operation of supplying the micro-plate in the micro-plate supplying and containing apparatus according to Embodiment 2 of the invention.

FIG. 28 is an operation explanatory view of operation of containing a micro-plate in the micro-plate supplying and containing apparatus according to Embodiment 2 of the invention.

FIG. 29 is an operation explanatory view of the operation of containing the micro-plate in the micro-plate supplying and containing apparatus according to Embodiment 2 of the invention.

### BEST MODE FOR CARRYING OUT THE INVENTION

### EMBODIMENT 1

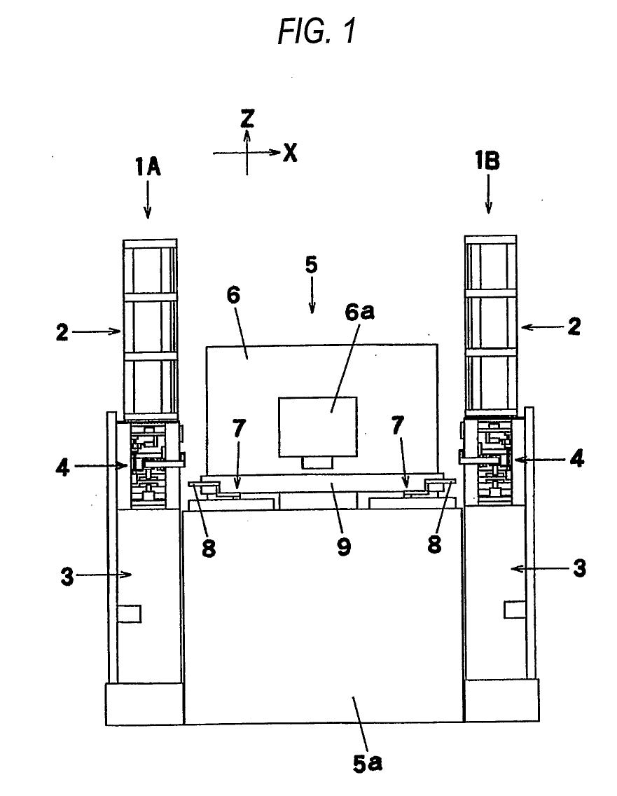

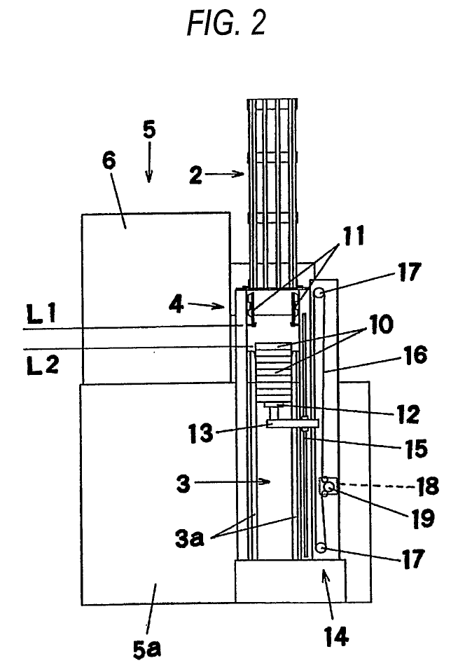

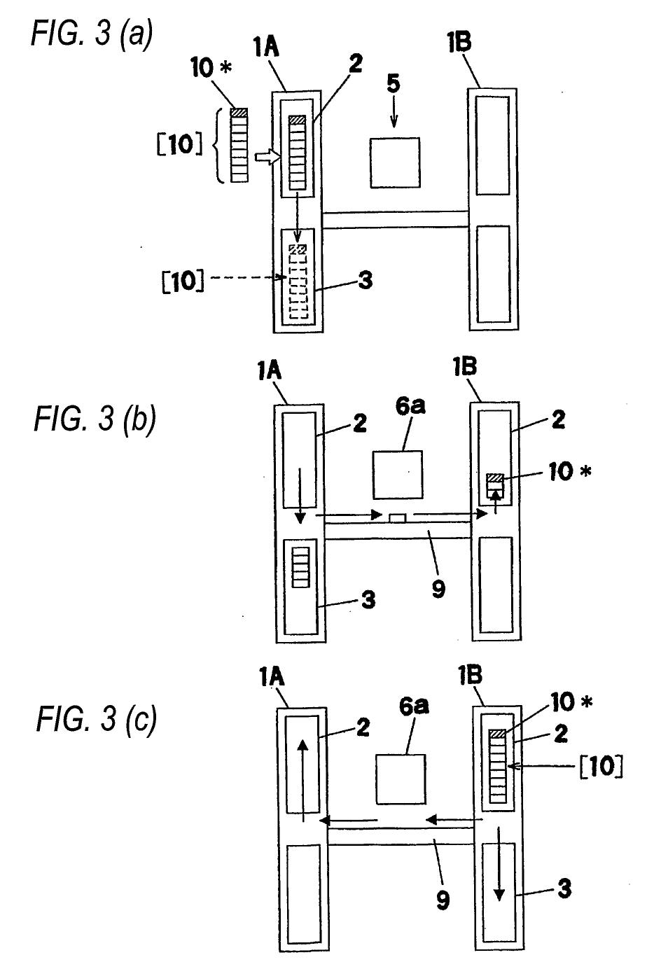

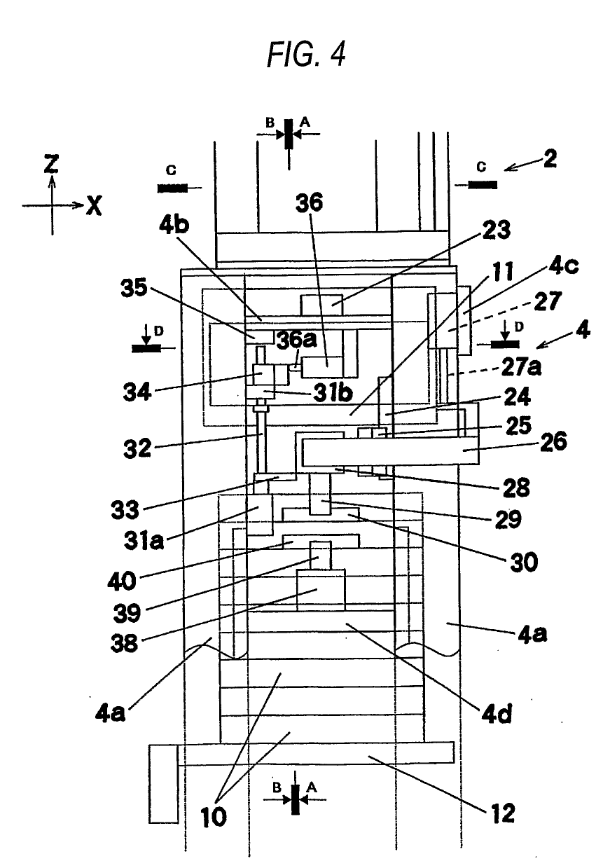

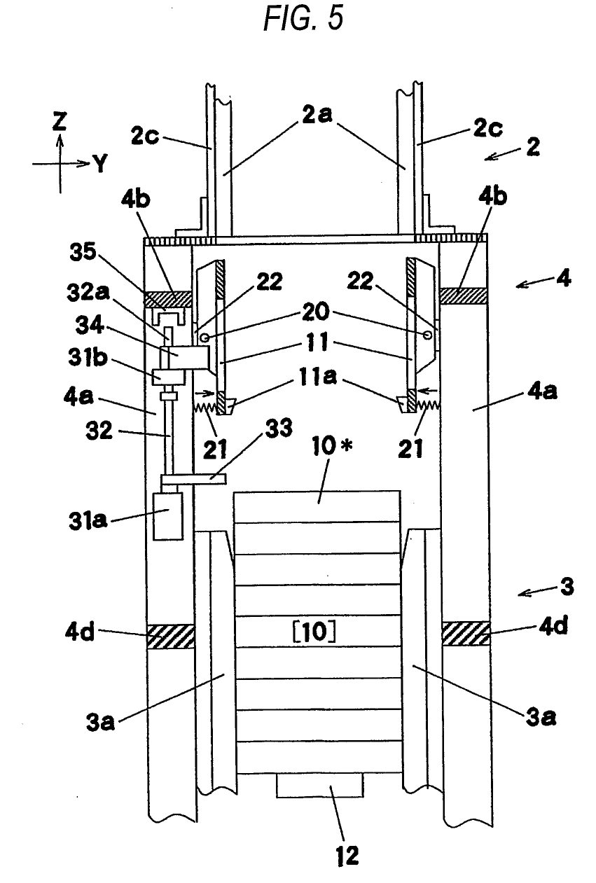

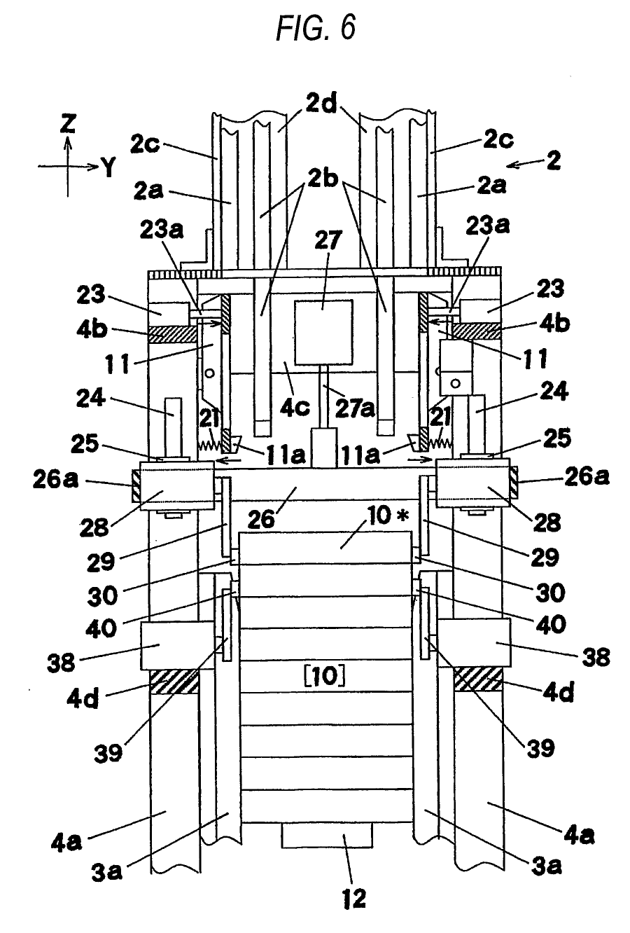

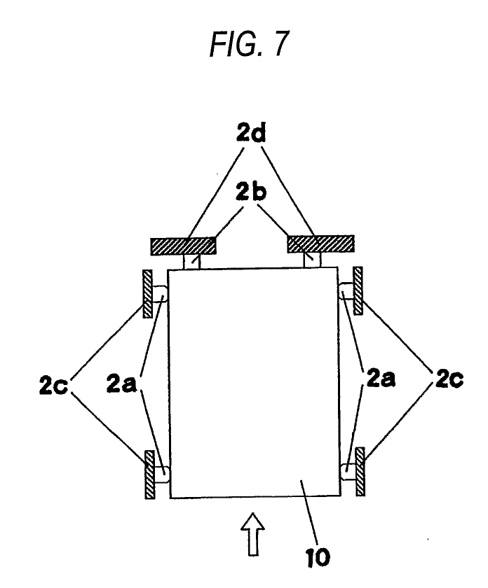

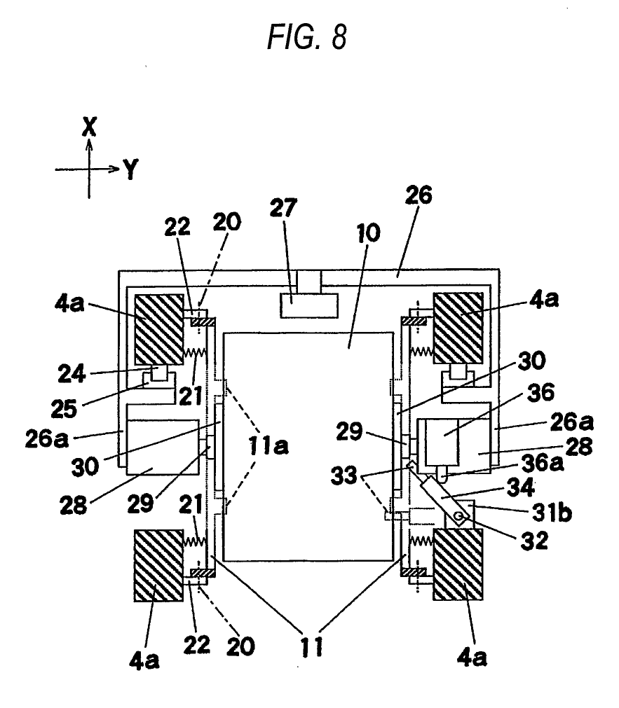

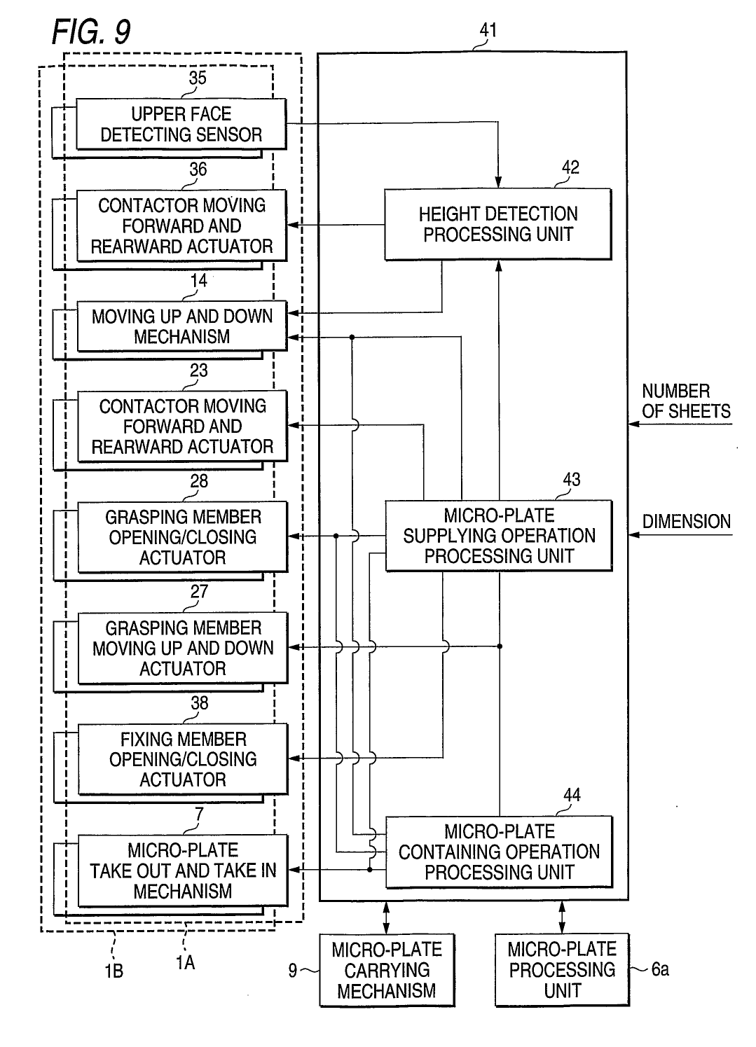

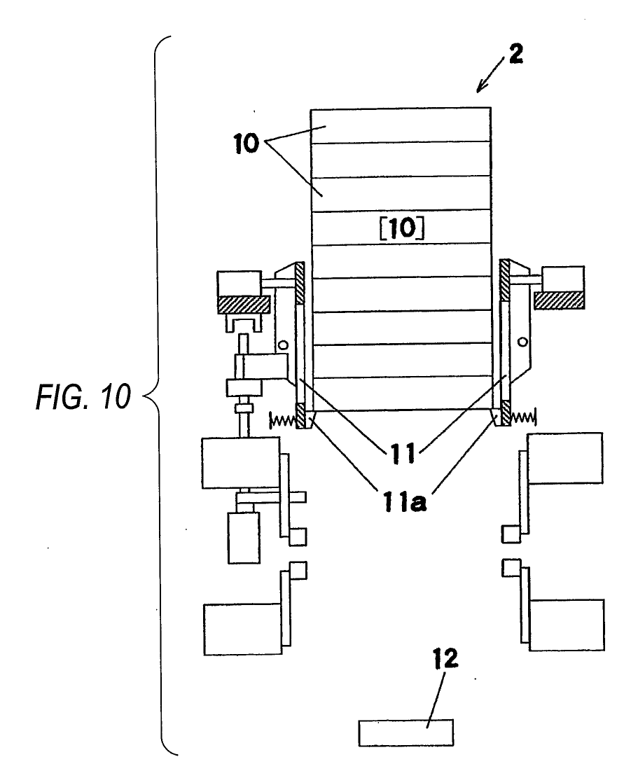

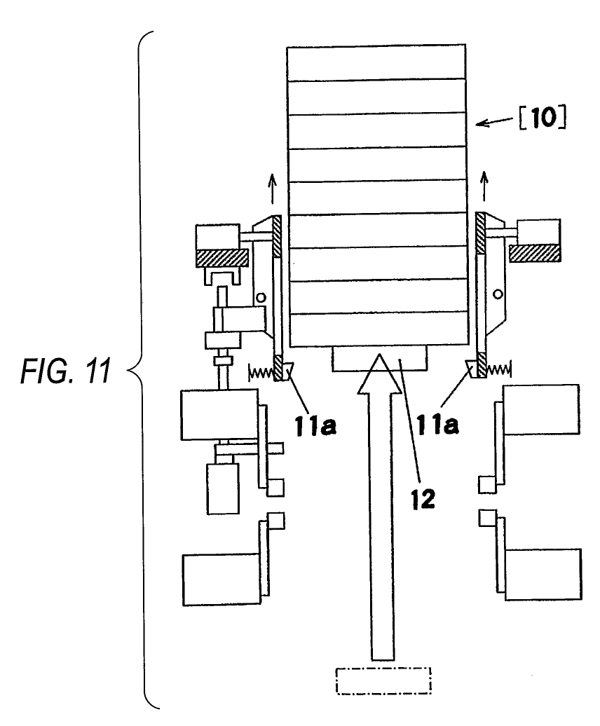

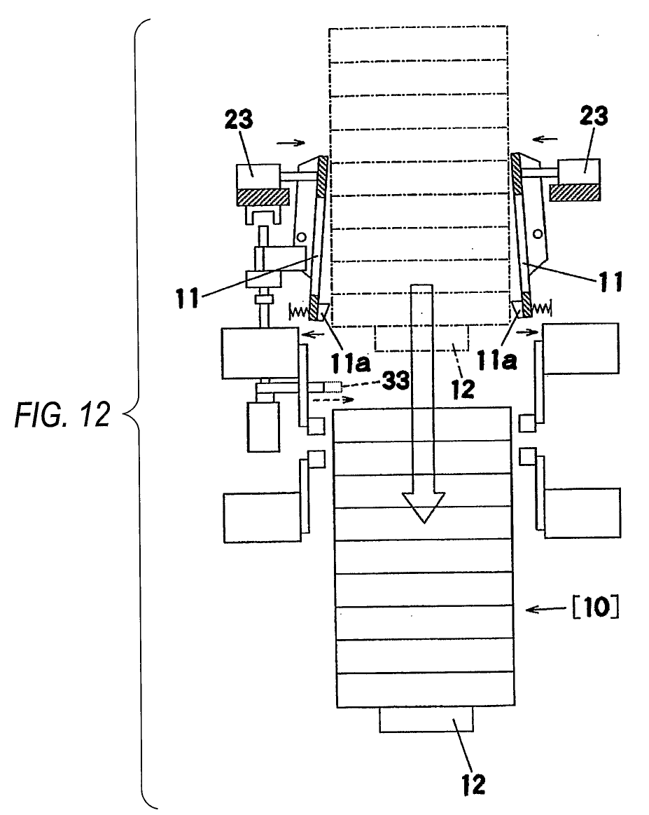

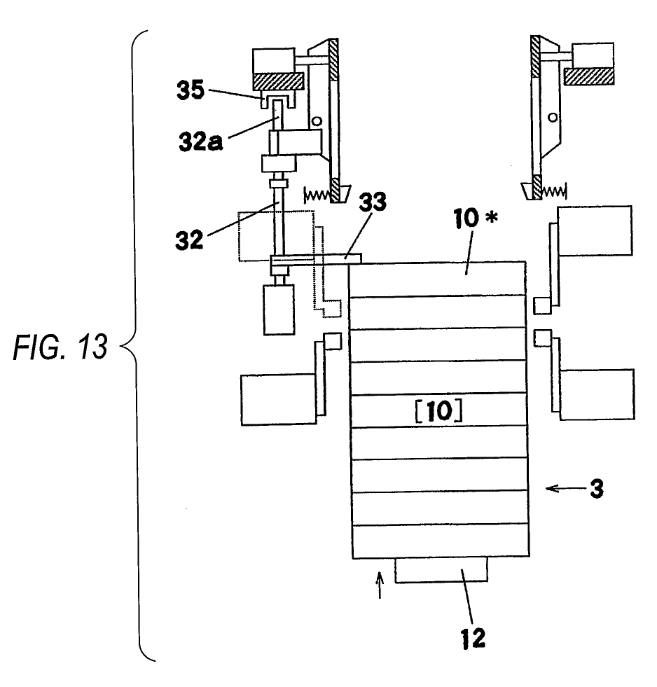

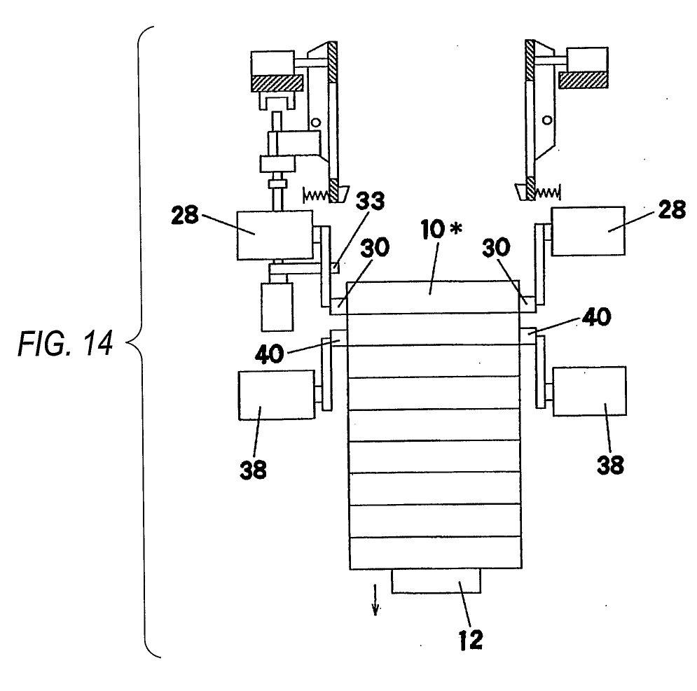

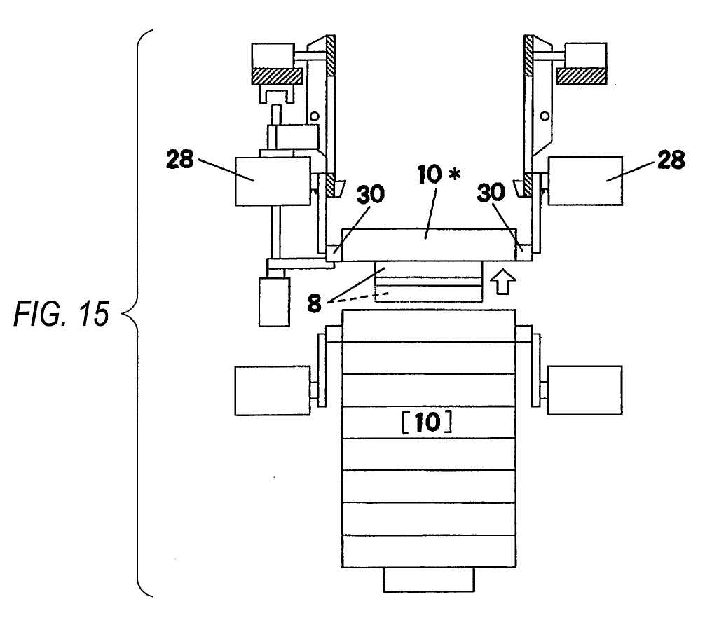

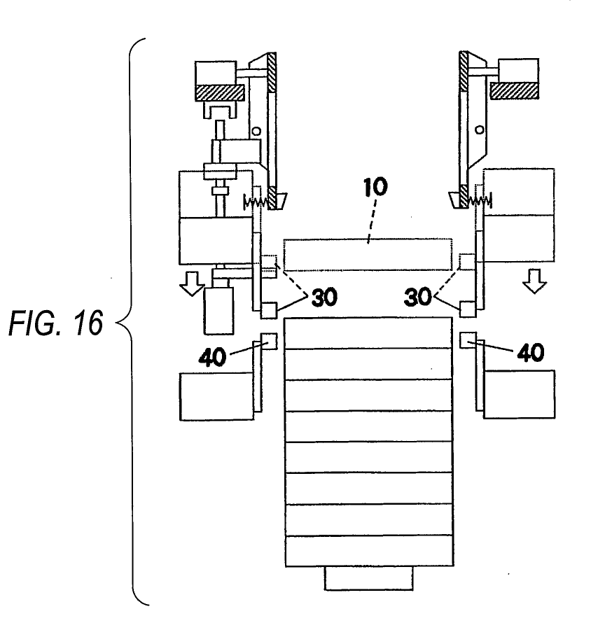

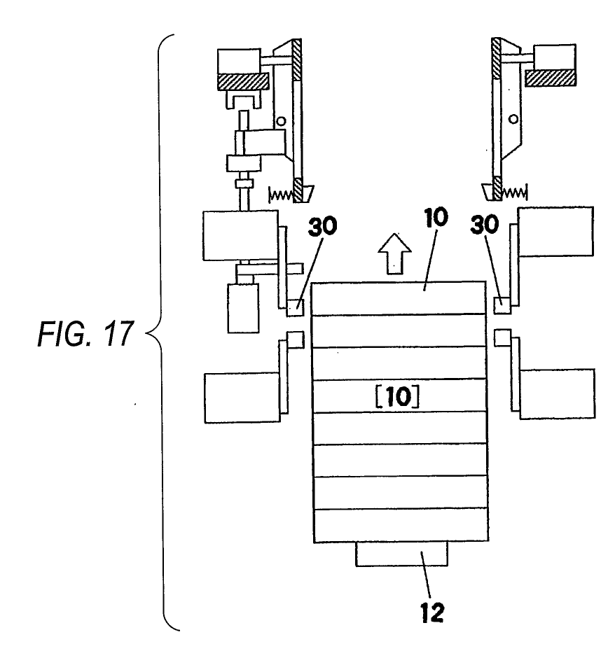

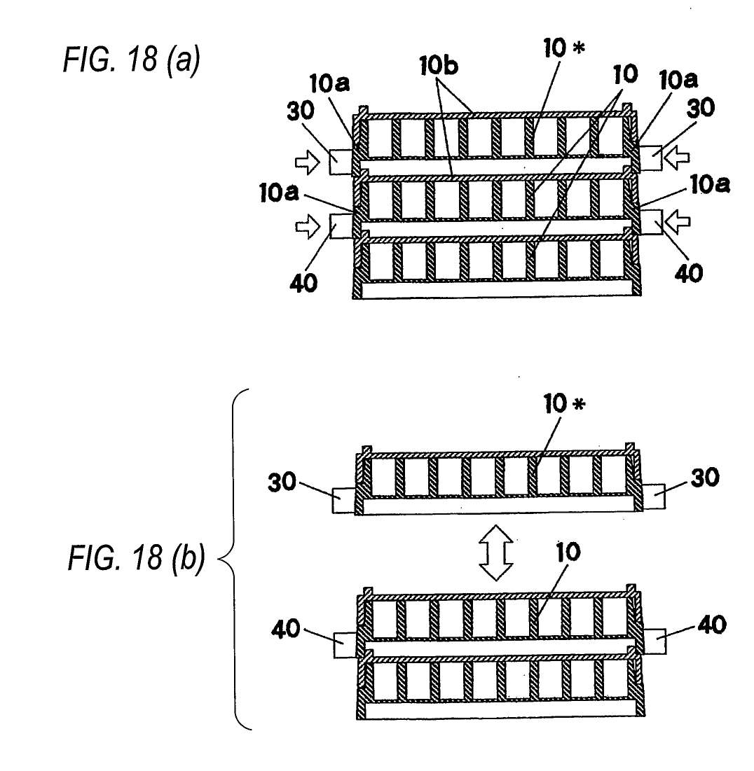

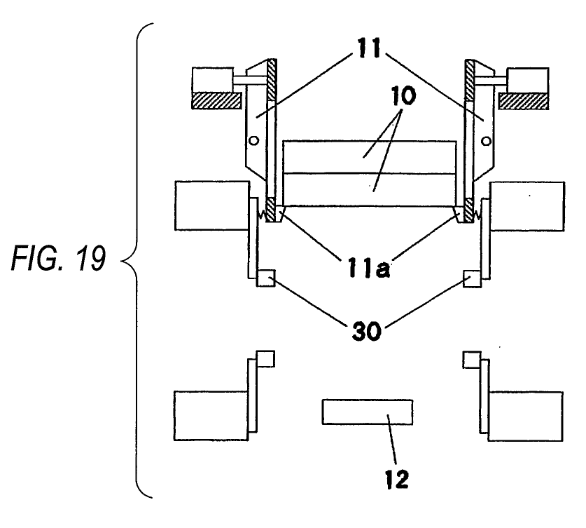

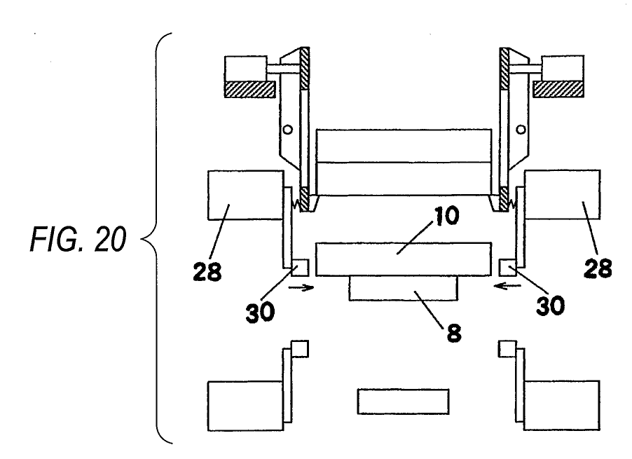

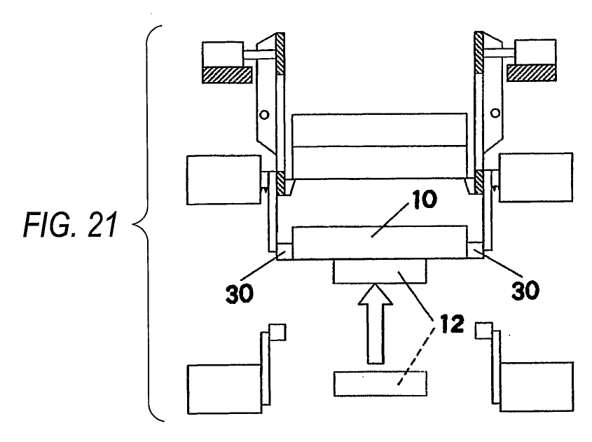

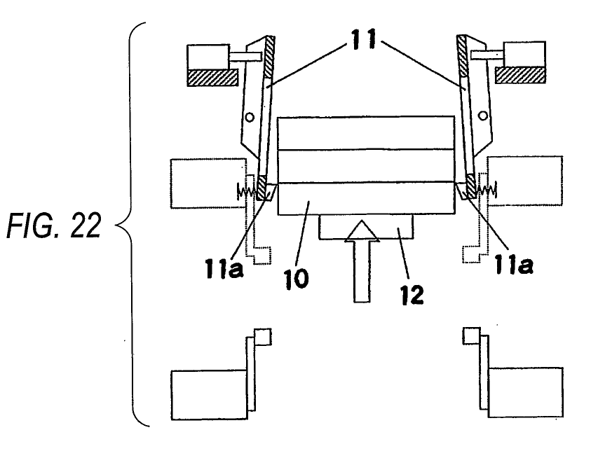

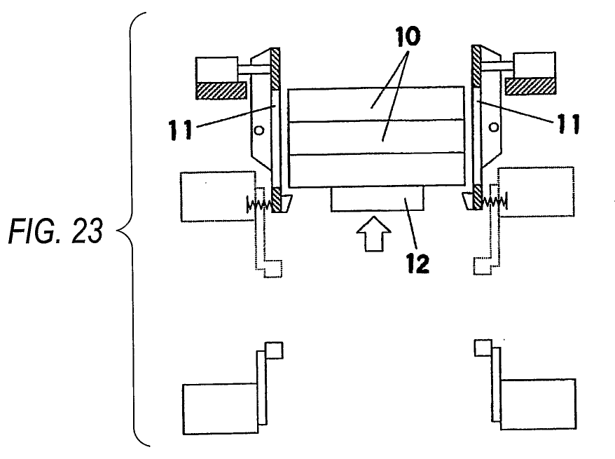

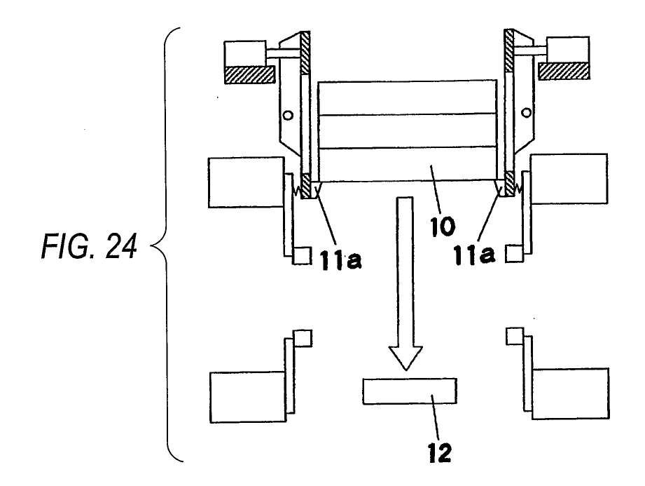

FIG. 1 is a front view of a working apparatus of Embodiment 1 of the invention, FIG. 2 is a side view of the working apparatus of Embodiment 1 of the invention, FIG. 3 illustrates explanatory views of total operation of the working apparatus according to Embodiment 1 of the invention, FIG. 4 is a side view of an essential portion of a micro-plate supplying and containing apparatus according to Embodiment 1 of the invention, FIG. 5, FIG. 6, FIG. 7, FIG. 8 are partial sectional views of the micro-plate supplying and containing apparatus according to Embodiment 1 of the invention, FIG. 9 is a block diagram showing a constitution of a control system of the working apparatus according to Embodiment 1 of the invention, FIG. 10, FIG. 11, FIG. 12, FIG. 13, FIG. 14, FIG. 15, FIG. 16, FIG. 17, FIG. 18 are operation explanatory views of operation of supplying a micro-plate in the micro-plate supplying and containing apparatus according to Embodiment 1 of the invention, and FIG. 19, FIG. 20, FIG. 21, FIG. 22, FIG. 23, FIG. 24 are operation explanatory views of micro-plate containing operation of the micro-plate supplying and containing apparatus according to Embodiment 1 of the invention.

First, a total constitution of a working apparatus will be explained in reference to FIG. 1, FIG. 2. The working apparatus is provided with a function of executing various processings of a dividedly injecting processing of injecting and discharging a specimen or a reagent to and from a well by constituting an object by a micro-plate provided with a plurality of wells for containing a liquid. As shown by FIG. 1, the working apparatus is constructed by a constitution of attaching micro-plate supplying and containing apparatus 1A, 1B to both sides of a working apparatus 5 for carrying out the above-described processings.

The micro-plate supplying and containing apparatus 1A, 1B are provided with a function of supplying a micro-plate 10 (refer to FIG. 2) constituting a processing object to the working apparatus 5 and recovering and containing the micro-plate 10 processed by the working apparatus 5. The micro-plate supplying and containing apparatus 1A, 1B are constituted by a structure of being symmetric with each other relative to the working apparatus 5, the micro-plate 10 supplied from the micro-plate supplying and containing apparatus 1A can be supplied by the micro-plate supplying and containing apparatus 1B, and contrary thereto, the micro-plate 10 supplied from the micro-plate supplying and containing apparatus 1B can be contained by the micro-plate supplying and containing apparatus 1A.

Naturally, in using the micro-plate supplying and containing apparatus 1A, 1B having such a constitution, only the function of supplying or only the function of containing the micro-plate 10 may be used. In this case, the micro-plate supplying and containing apparatus 1A, 1B function as micro-plate supplying apparatus or micro-plate containing apparatus.

Further, the embodiment constitutes an object by a micro-plate having a lid in a state of being mounted with a lid 10b at an upper face thereof as shown by FIG. 18 as the micro-plate 10, the micro-plates 10 are stacked in a state of covering an upper face of a well by the lid 10b and separated individually from a stacked state. Naturally, the micro-plate 10 in a state of removing the lid 10b may constitute a supplying or containing object.

The micro-plate supplying and containing apparatus 1A, 1B are constructed by a constitution of arranging first stock portions 2 and second stock portions 3 capable of containing pluralities of the micro-plates respectively in the stacked state in series in an up and down direction and there a plate take out and take in mechanism 4 is arranged at a middle of the first stock portion 2 and the second stock portion 3. Further, the working apparatus 5 is constituted by a structure of mounting a processing apparatus 6 above a base 5a, and a micro-plate carrying mechanism 9 for carrying the micro-plate 10 to a working unit 6a is arranged above the base 5a in a horizontal direction.

Micro-plate carrying out mechanisms 7 are arranged at two left and right ends of the micro-plate carrying mechanism 9 and the micro-plate carrying out mechanism 7 receives and delivers the micro-plate 10 between the plate take out and take in mechanism 4 and the micro-plate carrying mechanism 9 by a support table 8 capable of moving forward and rearward in the horizontal direction and moving up and down. That is, the micro-plate carrying mechanism 7 advances the support table 8 into the plate take out and take in mechanism 4 to dispose on a lower side of the micro-plate 10 constituting a take out object and successively moves up the support table 8 to support the micro-plate 10 from a lower side.

Further, by moving rearward the support table 8 from the plate take out and take in mechanism 4 to move downward, the micro-plate 10 taken out from the plate take out and take in mechanism 4 is delivered to the micro-plate carrying mechanism 9. Further, by making the micro-plate carrying out mechanism 7 execute operation in an order reverse to the above-described, the micro-plate 10 received from the micro-plate carrying mechanism 9 can be delivered to the plate take out and take in mechanism 4.

As shown by FIG. 2, a first support member 11 capable of opening and closing by an opening and closing mechanism, mentioned later, is provided at a lower portion of the first stock portion 2. The first support member 11 can support a plurality of the micro-plates 10 contained in the first stock portion 2 in a stacked state and release support of the micro-plates 10 to pass in an up and down direction, as described later. That is, the first stock portion is constructed by a constitution of including a support release mechanism for changing a state of the first support member 11 into a state of being unable to support the micro-plate 10.

The second stock portion 3 having a long vertical length arranged on the lower side of the first stock portion 2 in series in the up and down direction is constituted by a structure of capable of guiding and containing the micro-plates 10 in the stacked state by a width direction restricting guide member 3a arranged in the up and down direction, and is provided with a second support member 12 for supporting the plurality of micro-plates 10 in the stacked state at inside of the second stock portion 3.

The second support member 12 is guided in the up and down direction by a guide 15 via a moving up and down member 13. The moving up and down mechanism 14 is constructed by a constitution of aligning a belt 16 expanded by two up and down pulleys 17 to be able to travel in the up and down direction by a pulley 19 coupled to a rotating shaft of a motor 18 and coupling the moving up and down member 13 to the belt 16. By driving to rotate the motor 18, the second support member 12 is driven to move up and down via the moving up and down member 13 and is moved up and down along with the micro-plates 10 in the stacked state supported at an upper face thereof.

A height level L1 of a lower end portion of the first support member 11 becomes a support level L1 for locking and supporting the micro-plate 10 at a lowermost stage of the micro-plate 10 in the stacked state contained in the first stock portion 2 (referred to a locking member 11a shown in FIG. 4). Further, a height level L2 shown in FIG. 2 constitutes a micro-plate carry out level L2 for carrying out the micro-plate 10 at a uppermost stage of the micro-plate 10 in the stacked state stocked at the second stock portion 3.

Further, operation of carrying out and recovering the micro-plate, mentioned later, is carried out by advancing the support table 8 provided at the micro-plate carry out mechanism 7 to a space between L1 through L2. The micro-plate carry out mechanism 7 and a grasp mechanism, micro-plate separating means, mentioned later, constitute micro-plate carrying means for carrying the micro-plate 10 disposed at the micro-plate carry out level L2 of the second stock portion 3 to outside and receiving to deliver the micro-plate 10 to the second support member 12 from outside.

Next, an explanation will be given of a total operation of supplying and containing a micro-plate carried out at the working apparatus 5 in reference to FIG. 3. Rectangular frames 1A, 1B shown on both sides of the working apparatus 5 in FIG. 3 respectively show stock portions provided at the micro-plate supplying and containing apparatus 1A, 1B. That is, the stock portions each is constructed by a constitution of arranging the first stock portions 2 having a long vertical length for containing the plurality of micro-plates 10 in the stacked state and the second stock portions 3 having a long vertical length arranged on a lower side of the first stock portions 2 in series in the up and down direction.

As shown by FIG. 3(a), the first stock portion 2 of the micro-plate supplying and containing apparatus 1A is mounted with a plate stacked member (hereinafter, abbreviated as stacked member [10]) stacked with the plurality of microplates 10. Further, the micro-plate 10 disposed at the uppermost stage in the stacked member [10] is attached with notation 10* to differentiate from others. First, the stacked member [10] mounted to the first stock portion 2 is moved to the second stock portion 3 in the stacked state by a micro-plate moving up and down means, mentioned later.

Further, the micro-plate 10* at the uppermost stage is disposed at the micro-plate carry out level L2 by moving up and down the stacked member [10] in the second stock portion 3 and the micro-plate 10* is taken out by the micro-plate carrying out mechanism 7 shown in FIG. 1. That is, there is constructed a constitution having the micro-plate moving up and down means for moving down the plurality of micro-plates 10 stocked at the first stock portion 2 to move to the second stock portion 3 and disposing the micro-plate 10* at the uppermost stage in the plurality of micro-plates 10 at the micro-plate carrying out level L2 set at the second stock portion 3.

As shown by FIG. 3b, the taken-out micro-plate 10 is carried to an operating position by the working unit 6a by the micro-plate carrying mechanism 9, where predetermined processing operation of dividedly injecting processing or the like is carried out by the working unit 6a. The micro-plate 10 finished with the processing operation is carried to a right side again by the micro-plate carrying mechanism 9 and is delivered to the micro-plate supplying and containing apparatus 1B by the micro-plate carrying out mechanism 7.

The delivered micro-plate 10 is successively taken to the first stock portion 2 of the micro-plate supplying and containing apparatus 1B from the lower side and as shown by FIG. 3(c), all the micro-plates 10 of the stacked member [10] are contained at the first stock portion 2 to finish one processing. In this case, the micro-plate 10* disposed at the uppermost stage in a state of being mounted to the first stock portion 2 of the micro-plate supplying and containing apparatus 1A is similarly disposed at the uppermost stage even in a state of being contained in the first stock portion 2 of the micro-plate supplying and containing apparatus 1B.

Further, thereafter, in executing a successive processing, in the micro-plate supplying and containing apparatus 1B, the stacked member [10] is moved down from the first stock portion 2 to the second stock portion 3, the micro-plate 10 of the uppermost stage is similarly disposed at the micro-plate carry out level L2 and is taken out by the micro-plate carrying out mechanism 7. Further, the micro-plate 10 is carried to the working unit 6a by the micro-plate carrying mechanism 9 to execute a predetermined processing operation, thereafter, carried to the micro-plate supplying and containing apparatus 1A and is successively contained similarly at the first stock portion 2 of the micro-plate supplying and containing apparatus 1A.

Further, although in the example shown in FIG. 1, the micro-plate carrying out mechanism 7 is integrally provided with the working apparatus 5, the function of the micro-plate carrying out mechanism 7 may be integrated to the micro-plate supplying and containing apparatus 1A, 1B, explained below. Further, although the micro-plate 10 is carried to the working unit 6a via the micro-plate carrying mechanism 9, the micro-plate 10 may be carried directly to the operating position by the working unit 6a by the micro-plate carrying mechanism 7.

Next, an explanation will be given of detailed structures of the micro-plate supplying and containing apparatus 1A, 1B in reference to FIG. 4 through FIG. 8. FIG. 5, FIG. 6, FIG. 7, FIG. 8 respectively show A-A section, B-B section, C-C section, D-D section of FIG. 4. First, a sectional shape of the first stock portion 2 will be explained in reference to FIG. 7. As shown by FIG. 7, the first stock portion 2 is constituted by a structure of arranging frame plates 2c, 2d in the up and down direction by an arrangement of surrounding the micro-plate 10 from surroundings in three directions in plane view of the micro-plate 10, and the micro-plate 10 can be mounted to and taken out from an inner portion thereof from a face on this side (refer to arrow mark). Width direction restricting guide members 2a, charging direction position restricting guide members 2b are attached in the up and down direction and the micro-plate 10 contained in the first stock member is moved up and down along the guides.

Next, a structure of the plate take out and take in mechanism 4 will be explained. The plate take out and take in mechanism 4 is constituted by a structure of attaching mechanism elements, explained below, to support column members 4a arranged at four corners thereof (refer to FIG. 8). In the support column members 4a, two piece thereof opposed to each other in X direction are connected by connecting frames 4b at upper portions thereof and connecting frames 4d at lower portions thereof respectively in horizontal direction (refer to FIG. 5), and two pieces thereof opposed to each other in Y direction are connected by a connecting frame 4c arranged in Y direction (refer to FIG. 4).

First, the first support member 11 will be explained. In FIG. 8, a pair of the first support members 11 are arranged to be opposed to each other on inner sides in Y direction of the support column members 4a. The support column member 4a is fixedly attached with a bracket 22 and at the bracket 22, the first support member 11 is axially supported by a horizontal support pin 20 to be made to be able to pivot around the support pin 20. As shown by FIG. 4, FIG. 5, the first support member 11 is a member having a frame-like shape disposed in XZ plane and a lower end portion of the first support member 11 is provided with a locking member 11a projected in an inner side direction. The locking member 11a is urged in the inner side direction by a compression spring 21 interposed between the support column member 4a and a back face of the first support member 11 and the pair of locking members 11a opposed to each other are opened and closed in the form of being pivoted around the support pins 20.

As shown by FIG. 6, the connecting frame 4b is arranged with a support release actuator 23 for bringing a rod 23a into contact with the upper portion of the first support member 11, and by projecting the rod 23a, the locking member 11a is moved in an outer side direction against an urge force of the compression coil spring 21. In a state in which the rod 23a is brought into a retracted state, the upper portion of the first support member 11 is pressed to the rod 23a by the urge force of the compression spring 21 and the first support member 11 is brought into an erected attitude, as shown by FIG. 8, the locking member 11a can support the stacked member [10] contained in the first stock portion 2 by locking a lower face of the micro-plate 10.

Further, by projecting the rod 23a, the stacked member [10] in the first stock portion 2 can be moved to the second stock portion 3 on the lower side by moving the locking member 11a to the outer side against the urge force of the compression spring 21. Therefore, the support release actuator 23 constitutes a support release mechanism for changing the state of the first support member 11 into a state in which the micro-plate 10 cannot be supported.

Further, inner side faces of the locking members 11a opposed to each other are made to constitute taper faces upper sides of which are narrower than lower sides thereof, thereby, the micro-plate 10 can be passed to the first stock portion 2 from the lower side. That is, when the micro-plate 10 disposed on the lower side of the first support member 11 is pressed up to the upper side, the micro-plate 10 is brought into contact with the locking member 11a to press to expand the locking member 11a against the urge force of the compression spring 21.

Thereby, the micro-plate 10 is moved to the upper side of the locking member 11a. Further, by moving the locking members 11a to the inner sides by the compression springs 21 to close the locking members 11a opposed to each other, the lower face of the micro-plate 10 is locked by upper faces of the locking members 11a. In this way, the micro-plate 10 which is taken in from outside can be moved into the first stock portion 2 to contain (refer to FIG. 29). At this occasion, the above-described operation can be carried out even in a state in which a plurality of micro-plates 10 have already been contained in the first stock portion, thereby, the micro-plates 10 which are taken from outside can be pressed up from the lower side into the first stock portion 2 to be successively contained.

Next, a mechanism of grasping the micro-plate 10 will be explained. The grasping mechanism is provided with the function of pressing a grasping member to a side face of the micro-plate 10* at the uppermost stage disposed at the micro-plate carry out level L2 in the second stock portion 3. In FIG. 6, FIG. 8, at a side face of the support column member 4a, a guide rail 24 is arranged in the up and down direction and a slider 25 slidably fitted to the guide rail 24 is coupled to a connecting member 26.

The connecting member 26 is a frame member substantially in a gate-like shape and an extended portion 26a constituted by extending the connecting member 26 in X direction is fixedly attached with a grasping member opening/closing actuator 28. As shown by FIG. 6, a rod of the grasping member opening/closing actuator 28 is coupled with a grasping member 30 via a vertical arm 29 in a shape of a vertical plate. By driving the grasping member opening/closing actuator 28 in a state in which the grasp member 30 coincides with a height level of the micro-plate 10* at the uppermost stage of the stacked member [10] supported by the second support member 12, the grasping member 30 is pressed to a side face of the micro-plate 10* to grasp the micro-plate 10* (refer to FIG. 18). That is, the grasping member 30 and the grasping member opening/closing actuator 28 constitute a grasping mechanism for pressing the grasping member 30 to the side face of the micro-plate 10* at the uppermost stage disposed at the micro-plate carry out level L2 to grasp the micro-plate 10*.

The connecting member 26 is coupled to a rod 27a of a grasping member moving up and down actuator 27 arranged on an inner side of the connecting frame 4c in a vertical attitude. By extracting and retracting the rod 27a by driving the grasping member moving up and down actuator 27, the connecting member 26 is moved up and down along with the grasping member opening/closing actuator 28, a vertical arm 29 and the grasping member 30. That is, the grasping member moving up and down actuator 27, the guide rail 24, the slider 25 and the connecting member 26 constitute a grasping member moving up and down mechanism.

By moving up the grasping member moving up and down actuator 27 in a state of grasping the micro-plate 10* by the grasping member 30, the grasping member 30 grasping the micro-plate 10* is moved in a direction of being remote from the stacked member [10] supported by the second support member 12, thereby, the micro-plate 10* at the uppermost stage is separated from the micro-plate 10 at a successive stage. Therefore, the grasping member moving up and down mechanism for moving up and down the grasping member 30 constitutes micro-plate separating means for separating the micro-plate 10* at the uppermost stage from the micro-plate 10 at the successive stage by relatively moving the second support member 12 and the grasping member 30 grasping the micro-plate 10 in directions of being remote from each other.

Further, in order to separate the micro-plate 10* at the uppermost stage, instead of moving up the grasping member 30 grasping the micro-plate 10, the second support member 12 may be moved down along with the stacked member [10] at the successive stage or lower by driving the moving up and down mechanism 14. In this case, the moving up and down mechanism 14 functions as micro-plate separating means.

Further, the micro-plate 10 separated by the micro-plate separating means is supported by the support table 8 of the micro-plate carrying out mechanism 7 and is carried out to outside of the plate take out and take in mechanism 4. Therefore, the micro-plate carrying out mechanism 7 constitutes micro-plate carrying out means for carrying out the micro-plate 10* at the uppermost stage separated by the above-described micro-plate separating means. Further, the micro-plate separating means is constructed by a constitution having the support table 8 for supporting the micro-plate 10* at the uppermost stage by advancing to between the micro-plate 10* at the uppermost stage separated by the micro-plate separating means and the micro-plate 10 at the successive stage.

In FIG. 6, an upper face of the connecting frame 4d is arranged with a fixing member opening/closing actuator 38 in a horizontal attitude, and a rod of the fixing member opening/closing actuator 38 is coupled to a fixing member 40 via a vertical arm 39 in a shape of a vertical plate. By driving the fixing member opening/closing actuator 38 in a state in which the fixing member 40 is disposed at a height level of the micro-plate 10 at a stage successive to the micro-plate 10* at the uppermost stage of the stacked member [10], both side faces of the micro-plate 10 at the successive stage are pressed by the fixing members 40 to fix a position thereof (refer to FIG. 18).

The position is fixed when the micro-plate 10* at the uppermost stage is separated by the grasping member 30. Thereby, there is prevented a drawback of a positional shift or the like from being brought about by making an attitude of the micro-plate 10 at the successive stage or lower unstable in the separating operation. That is, according to the embodiment, there is constructed a constitution having a fixing mechanism of temporarily fixing the micro-plate 10 at the successive stage when the micro-plate 10* at the uppermost stage is separated.

Next, an explanation will be given of upper face detecting means for detecting the upper face of the micro-plate 10* at the uppermost stage in the plurality of micro-plates 10 supported above the second support member 12 in the stacked state in reference to FIG. 5, FIG. 8. In FIG. 5, a side face of the support column member 4a (disposed on a right lower side in FIG. 8) is arranged with bearing members 31a, 31b for axially supporting a shaft member 32 rotatably and slidably. The shaft member 32 is provided with a contactor 33 in a direction of being extended to an inner side from a position right above the bearing member 31a and a pivoting lever 34 in a direction of being extended to an inner side from a position right above the bearing member 31b to be respectively fixedly attached to the shaft member 32. Both of the contactor 33 and the pivoting lever 34 are made to be turnable around the shaft member 32 and slidable in the up and down direction.

As shown by FIG. 8, a rod 36a of a contactor moving forward and rearward actuator 36 (also refer to FIG. 4) coupled to a lower face of the connecting frame 4b is brought into contact with a side face of the pivoting lever 34. By projecting the rod 36a by driving the contactor moving forward and rearward actuator 36, the pivoting lever 34 is moved to rotate the shaft member 32 around an axis thereof. Thereby, also the contactor 33 is pivoted similarly around the axis, a front end portion thereof is extended to an upper side of the stacked member [10] supported by the second support member 12 and the contactor 33 is made to move forward to a position capable of being brought into contact with contact with the micro-plate 10.

Further, when the second support member 12 is moved up under the state, the upper face of the micro-plate 10* at the uppermost stage is brought into contact with the contactor 33 to press down the upper face, and the shaft member 32 is pressed up by the contactor 33 to slide to the upper side. A lower face of the connecting frame 4b on the upper side of the shaft member 32 is arranged with an upper face detecting sensor 35, and by advancing a detecting end portion 32a at an upper end of the shaft member 32 into a detecting range of the upper face detecting sensor 35, the upper face detecting sensor 35 detects the detecting end portion 32a.

Thereby, it is detected that a height of the upper face of the micro-plate 10* at the uppermost stage reaches a previously set detecting height level. The detecting height level is related to the micro-plate carry out level L2, and by controlling to operate to move up and down the moving up and down mechanism 14 by constituting a reference by the detecting height level, the micro-plate 10* at the uppermost stage constituting a take out object is disposed at the micro-plate carry out level L2. Further, the pivoting lever 34 is urged always in the clockwise direction by an urge mechanism, not illustrated, and the contactor 33 is disposed at a position of not hampering the micro-plate 10 from moving up and down at normal time of not driving the contact moving forward and rearward actuator 36.

That is, according to the above-described constitution, the upper face detecting means is contact type detecting means having the contactor 33 brought into contact with the upper face of the micro-plate 10 and the contact type detecting means is constructed by a constitution of including the upper face detecting sensor 35 for detecting that the contactor 33 is moved to be brought into contact with the upper face of the micro-plate 10. Further, as described later, in operating to carry out the micro-plate 10, the micro-plate 10* at the uppermost stage is disposed at the micro-plate carry out level L2, that is, the grasping position by the grasping mechanism by making a controller 41, explained below, control the moving up and down mechanism 14 by constituting a reference by the height position of the second support member 12 when the upper face detecting means detects the upper face of the micro-plate 10.

Further, as the upper face detecting means, noncontact type detecting means (a reflection type sensor, a light shielding type sensor combined with a light emitting element and a light receiving element or the like) maybe used. However, the micro-plate is frequently made from a transparent material and therefore, it is also anticipated that the upper face of the micro-plate cannot accurately be detected by the noncontact type detecting means of an optical type. Therefore, as the upper detecting means, contact type detecting means capable of firmly detecting the upper face regardless of the material of the micro-plate as shown in the embodiment is preferable.

Next, a constitution of a control system will be explained in reference to FIG. 9. Further, the micro-plate supplying and containing apparatus 1A, 1B are provided with the same constitution, and in FIG. 9, only the constitution of the micro-plate supplying and containing apparatus 1A is clearly shown. In FIG. 9, the controller 41 is a control apparatus provided to the working apparatus 5 for controlling operation of respective elements of the micro-plate supplying and containing apparatus 1A, 1B along with a processing of controlling to operate the micro-plate carrying mechanism 9.

A height detection processing unit 42, a micro-plate supplying operation processing unit 43, and a micro-plate containing operation processing unit 44 show processing functions realized by executing processing programs stored to the controller 41. In carrying out the control processings, a number of sheets and a dimension (thickness dimension of micro-plate 10) of the micro-plate 10 constituting a processing object are previously inputted.

The height detection processing unit processes to detect the height of the micro-plate 10 by the above-described upper face detecting means. That is, the second support member 12 is moved up by controlling the moving up and down mechanism 14 in a state of advancing the contactor 33 to the detecting position by controlling the contactor moving forward and rearward actuator 36. Further, the upper face detecting sensor 35 detects that the upper face of the micro-plate 10* at the uppermost stage of the stacked member [10] held above the second support member 12 is brought into contact with the contactor 33, and the height position of the second support member 12 at this occasion is outputted as the height of the upper face. The processing of detecting the height is executed by an instruction of the micro-plate supplying operation processing unit 43, explained below, and a detecting result is transmitted to the micro-plate supplying operation processing unit 43.

The micro-plate supplying operation processing unit 43 executes the following operation processings by controlling the moving up and down mechanism 14, the support release actuator 23, the grasping member opening/closing actuator 28, the grasping member moving up and down actuator 27, the fixing member opening/closing actuator 38 and the micro-plate carrying out mechanism 7. That is, by controlling the moving up and down mechanism 14, there is executed operation of changing the first support member 11 into a state of being unable to support the micro-plate 10 by carrying out operation of switching to mount the plurality of micro-plates 10 supported by the support member 11 to the second support member 12 by moving up the second support member 12 and controlling the support release actuator 23.

Further, the micro-plate supplying operation processing unit 43 operates the support release mechanism and the moving up and down mechanism 14 to dispose the micro-plate 10* at the uppermost stage of the plurality of micro-plates 10 to the micro-plate carrying out level L2 by moving the plurality of micro-plates 10 which have been switched to mount by moving down the second support member 12 to the second stock portion 3 and controlling the height position of the second support member 12 based on a result of detecting the height by the height detecting processing unit 42 by controlling the moving up and down mechanism 14.

The micro-plate containing operation processing unit 44 executes to operate to contain the micro-plate 10 which has been carried in from outside to the plate take out and take in mechanism 4 by the micro-plate carrying out mechanism 7 from the lower side into the first stock portion 2 by controlling the moving up and down mechanism 14, the grasping member moving up and down actuator 27, the grasping member opening/closing actuator 28 and the micro-plate carrying out mechanism 7. That is, the micro-plate containing operation processing unit 44 operates the moving up and down mechanism 14 to support the micro-plate 10 delivered from the micro-plate carrying means by the first support member 11 by moving up the second support member 12.

The micro-plate supplying and containing apparatus 1A, 1B are constituted as described above, and in the following, an explanation will be given of operation of supplying the micro-plate for supplying the micro-plate 10 from the micro-plate supplying and containing apparatus 1A, 1B to the working apparatus 5 in reference to FIG. 10 through FIG. 18.

FIG. 10 shows a state immediately after charging the stacked member [10] stacked with the plurality of micro-plates 10. At this occasion, the first support member 11 is brought into a supporting state capable of supporting the micro-plate 10 and the lower face of the micro-plate 10 at the bottommost stage is locked by the locking member 11a. At this occasion, the second support member 12 disposed on the lower side is brought into a vacant state in which the micro-plate 10 is not supported.

Successively, a control instruction is issued from the micro-plate supplying operation processing unit 43 to the moving up and down mechanism 14. Thereby, as shown by FIG. 11, the second support member 12 is moved up to support the stacked member [10] supported by the first support member 11 from the lower side to move up until the micro-plate 10 at the bottommost stage is separated from the upper face of the locking member 11a to switch to mount the stacked member [10] to the second support member 12. Further, as shown by FIG. 12, the first support member 11 is opened by moving the locking member 11a to the outer side by driving the support release actuator 23 to bring about a state in which the stacked member [10] cannot be supported by the first support member 11. Successively, the second support member 12 is moved down, the stacked member [10] which has been switched to mount is moved to the second stock portion 3, thereafter, the support release actuator 23 is stopped to operate and the state of the first support member 11 is recovered to a state of capable of supporting the micro-plate 10.

Thereafter, the height detection processing is executed by the height detection processing unit 42. First, the contactor 33 is advanced to the detecting position and successively, search operation of moving up the second support member 12 at low speed is carried out. Thereafter, as shown by FIG. 13, the search operation is finished when the upper face detecting sensor 35 detects the detecting end portion 32a by moving the shaft member 32 by bringing the upper face of the micro-plate 10* at the uppermost stage into contact with the contactor 33. Further, the detecting result is transmitted to the micro-plate supplying operation processing unit 43 to recover to the processing by the micro-plate supply operation processing unit 43.

Successively, the height position of the micro-plate 10* at the uppermost stage is controlled to the position of capable of grasping the micro-plate 10 by the grasping member 30 based on machine parameters and dimension information (thickness dimension) of the micro-plate 10 which have previously been inputted. Further, as shown by FIG. 14, the grasping member 30 is closed by driving the grasping member opening/closing actuator 28, as shown by FIG. 18(a), the side face 10a of the micro-plate 10* at the uppermost stage is grasped, the micro-plate 10 at the successive stage is grasped to fix by driving the fixing member opening/closing actuator 38 to escape the contactor 33 to the normal position.

Thereafter, as shown by FIG. 15, the grasping member 30 is moved up by driving the grasping member moving up and down actuator 27. Thereby, as shown by FIG. 18(b), the micro-plate 10* at the uppermost stage is separated from the micro-plates 10 at the successive stage and lower. Successively, the support table 8 is advanced to the space between the lower face of the uppermost micro-plate 10* and the upper face of the micro-plate at the successive stage, and the lower face of the micro-plate 10 is supported by moving up the support table 8. Successively, the micro-plate 10 is released from being grasped by the grasping member 30 by driving the grasping member opening/closing actuator 28 and the micro-plate 10 is carried out by moving rearward the support table 8 as shown by FIG. 16. Further, the grasping member is moved down and the micro-plate 10 is released from being fixed by the fixing member 40.

Thereafter, as shown by FIG. 17, the second support member 12 is moved up by an amount of the thickness of the micro-plate 10, and the micro-plate 10 disposed at the successive stage is disposed at the position of grasping the micro-plate 10 by the grasping member 30, that is, the micro-plate carry level L2. Further, thereafter, the operation returns to the state shown in FIG. 14 and thereafter, operation similar to the above-described is repeatedly executed. Further, in stead of moving up the second support member 12 based on dimension information of the micro-plate 10, the height detection processing by the height detection processing unit 42 may be executed at respective times.

The above-described micro-plate supplying operation is constituted by a mode including a step of switching to mount the plurality of micro-plates 10 supported by the first support member 11 to the second support member 12 by moving up the second support member 12, a step of changing the state of the first support member 11 to the state in which the micro-plate 10 cannot be supported by operating the support release mechanism, a step of moving a plurality of micro-plates 10 which have been switched to mount by moving down the second support member 12 to the second stock portion 3, a step of disposing the micro-plate 10* at the uppermost stage of the plurality of micro-plates 10 to the micro-plate carry out level L2 by controlling the height position of the second support member 12, and a step of carrying the micro-plate 10 disposed at the micro-plate carry out level L2 to outside by the micro-plate carrying means.

Furthermore, the mode includes a step of detecting the upper face of the micro-plate 10* at the uppermost stage by the upper face detecting means after switching to mount the plurality of micro-plates 10 to the second support member 12, and the micro-plate 10* at the uppermost stage is disposed at the micro-plate carry out level L2 by controlling the moving up and down mechanism 14 by constituting the reference by the height position of the second support member 12 when the upper face of the micro-plate 10 is detected by the upper face detecting means.

Next, an explanation will be given of micro-plates containing operation for containing the micro-plate 10 delivered from outside into the first stop portion 2 in reference to FIG. 19 through FIG. 24. In FIG. 19, two sheets of the micro-plates 10 which have already been contained are locked by the locking member 11a at the first support member 11, and the second support member 12 and the grasping member 30 are at standby at predetermined height positions.

Successively, operation of containing a new micro-plate is started by the containing operation processing unit 44 and as shown by FIG. 20, the support table 8 supporting the micro-plate 10 at the upper face is advanced to between the first support member 11 and the second support member 12. Successively, when the micro-plate 10 is grasped by the grasping member by driving the grasping member opening/closing actuator 28, the support table 8 is escaped, successively, as shown by FIG. 21, the second support member 12 is moved up, the micro-plate 10* is released from being grasped by the grasping member 30 and the lower face of the micro-plate 10 is supported by the second support member 12. Thereby, the micro-plate 10 is delivered from the micro-plate carrying means to the second support member 12.

Furthermore, the second support member 12 is moved up, and as shown by FIG. 22, the upper end portion of the micro-plate 10 is moved up along the taper face of the locking member 11a. Thereby, the locking member 11a is pressed to expand the outer side, as shown by FIG. 23, by moving the micro-plate 10 to the upper side of the locking member 11a, the locking member 11a is recovered to a position of capable of locking the lower face of the micro-plate 10.

Successively, as shown by FIG. 24, by moving down the second support member 12, the micro-plate 10 is locked by the locking member 11a, and the micro-plate 10 which has newly been delivered is supported by the first support member 11 along with the micro-plates 10 which have already been contained. That is, the above-described micro-plate containing operation is constituted by a mode of including a step of delivering the micro-plate 10 to the second support member 12 by the micro-plate carrying means, and a step of supporting the micro-plate 10 delivered from the micro-plate carrying means by the support member 11 by moving up the second support member 12.

### EMBODIMENT 2

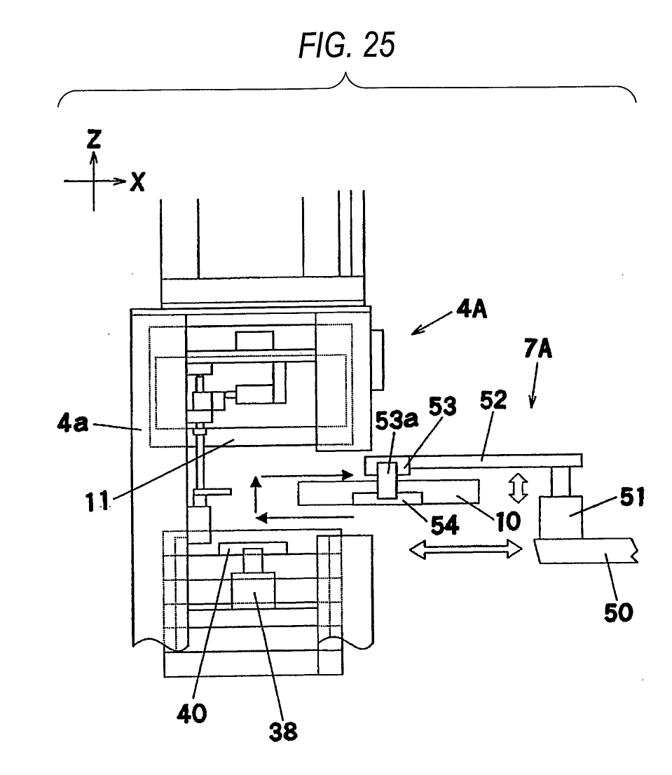

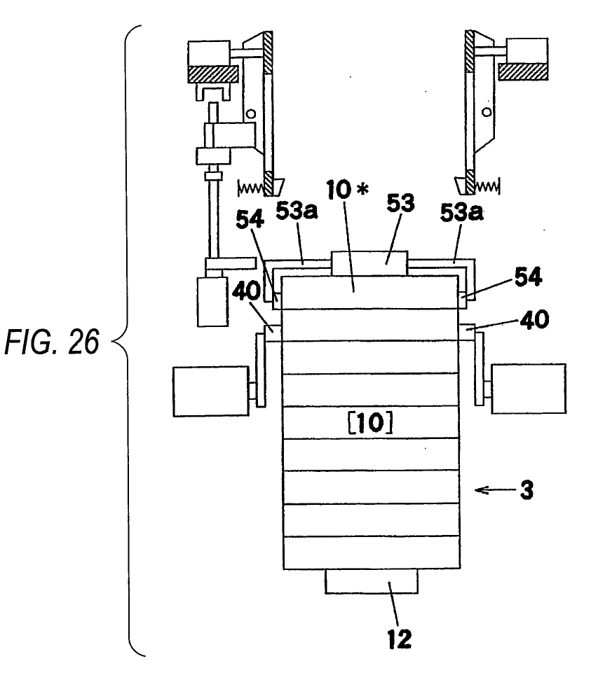

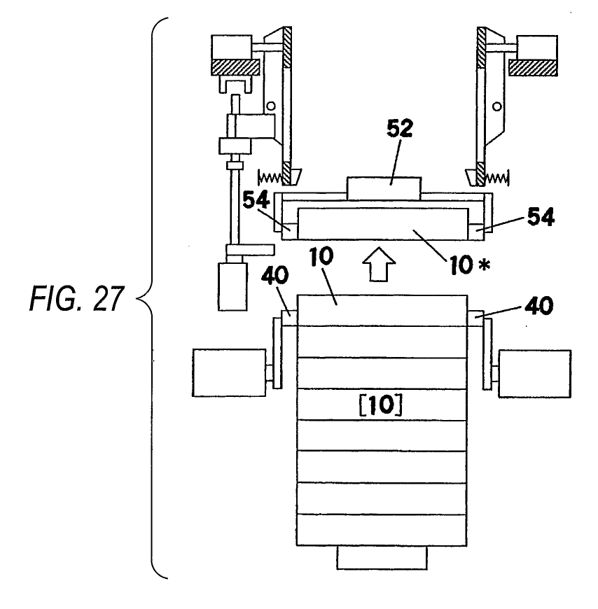

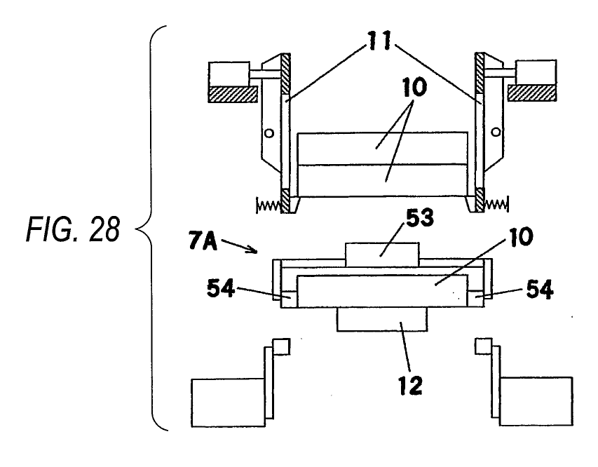

FIG. 25 is a side view of an essential portion of a micro-plate supplying and containing apparatus according to Embodiment 2 of the invention, FIG. 26, FIG. 27 are operation explanatory views of operation of supplying a micro-plate in the micro-plate supplying and containing apparatus according to Embodiment 2 of the invention and FIG. 28, FIG. 29 are operation explanatory views of operation of containing the micro-plate in the micro-plate supplying and containing apparatus according to Embodiment 2 of the invention.

FIG. 25 shows a micro-plate take out and take in mechanism 4A of the micro-plate containing and supplying apparatus according to Embodiment 2. The micro-plate take out and take in mechanism 4A is constructed by a constitution of excluding the mechanism of grasping the micro-plate 10 from the plate take out and take in mechanism 4 according to Embodiment 1. Further, a micro-plate carrying out mechanism 4A is constructed by a constitution of adding the grasping mechanism of the micro-plate 10 to the micro-plate carrying out mechanism 7 according to Embodiment 1. That is, Embodiment 2 is constructed by a constitution of providing the grasping mechanism of the micro-plate 10 to the micro-plate carrying out means.

As shown by FIG. 25, a moving up and down drive portion 51 is arranged above a horizontal moving mechanism 50 capable of moving horizontally in X direction, and a horizontal arm 52 is coupled to the moving up and down drive portion 51. A front end portion of the horizontal arm 52 is provided with a grasping member opening/closing mechanism 53 and a grasping member 54 is mounted to an opening/closing arm 53a of the grasping member opening/closing mechanism 53. The horizontal arm 52 is moved up and down by driving the moving up and down drive portion 51, thereby, the grasping member 54 is moved up and down. Therefore, the moving up and down drive portion 51 constitutes a grasping member moving up and down mechanism. Further, the opening/closing arm 53a is elongated or contracted in a horizontal direction by driving the grasping member opening/closing mechanism 53, thereby, the micro-plate 10 can be grasped by the grasping member 54. That is, the grasping member 54 and the grasping member opening/closing mechanism 53 constitute a grasping mechanism.

FIG. 26, FIG. 27 show operation of supplying a micro-plate. In FIG. 26, the second support member 12 of the second stock portion 3 holds the stacked member [10] and the micro-plate 10* at the uppermost stage is grasped by the grasping member 54 of the micro-plate carrying out mechanism 7A. Further, a position of the micro-plate 10 at the successive stage is fixed by the fixing member 40 of the fixing mechanism similar to that of Embodiment 1. Next, as shown by FIG. 27, the micro-plate 10 in a state of being grasped by the grasping member 54 is moved up to separate from the micro-plate 10 at the successive stage by driving the moving up and down drive portion 51 of the micro-plate carrying out mechanism 7A.

That is, Embodiment 2 is constituted by a mode in which micro-plate separating means is the grasping member moving up and down mechanism for moving up and down the grasping member 54 and the grasping member moving up and down mechanism is provided to micro-plate carrying means. Further, similar to Embodiment 1, there is constructed a constitution of having the fixing mechanism for temporarily fixing the micro-plate 10 at a successive stage when the micro-plate 10* at the uppermost stage is separated.

Next, operation of containing a micro-plate will be explained in reference to FIG. 28, FIG. 29. In FIG. 28, two sheets of the micro-plates 10 which have already been contained are held at the first support member 11, and the micro-plate 10 which newly constitutes a containing object is carried in by the micro-plate carrying out mechanism 7a in a state of being grasped by the grasping member 54 on the lower side of the first support member 11. Further, the lower face of the micro-plate 10 is supported by the second support member 12. Further, after supporting the micro-plate 10 by the second support member 12, the micro-plate 10 is released from being grasped by the grasping member 54 and the micro-plate carrying out mechanism 7A is escaped.

Further, as shown by FIG. 28, similar to the operation shown in FIG. 22 of Embodiment 1, the micro-plate 10 is pressed up by the second support member 12, the micro-plate 10 is moved to the upper side of the locking member 11a and is locked by the locking member 11a. Thereby, the micro-plates 10 carried in by the micro-plate carrying out mechanism 7A are successively contained at the first stock portion 2.

By the above-described constitution explained in Embodiments 1, 2, the micro-plate supplying and containing apparatus of the invention achieves an effect explained below. First, in the micro-plate supplying and containing apparatus 1A, 1B, by constituting the stock portion of containing the micro-plate 10 by arranging the first stock portion 2, the second stock portion 3 having long vertical lengths capable of containing the micro-plates in the stacked state in series in the up and down direction, the compact micro-plate supplying and containing apparatus having a small occupied area is realized, the micro-plates 10 can be mounted and taken out by an operator in the stacked state at the first stock portion 2 disposed at a height facilitating an operation attitude.

Thereby, when a test is carried out by constituting an object by a number of micro-plates, complicated operation which has been needed in the background art apparatus having a stock portion of amounting shelf type, that is, operation of mounting or taking out micro-plates individually to and from a position of mounting the micro-plates at a mounting shelf in supplying or recovering the micropalte can considerably be simplified and operating performance and operability can be promoted.

Further, there is adopted a constitution having the micro-plate moving up and down means enabling to support and release support of the micro-plates 10 in the stacked state at the first stock portion 2 and capable of supporting and moving up and down the micro-plates 10 in the stacked state in the second stock portion 3 and having the micro-plate carrying means capable of receiving and delivering the micro-plate disposed at the micro-plate carry out level L2 of the second stock portion 3 to and from outside.

Thereby, operation of supplying the micro-plates for moving the micro-plates 10 contained at the first stock portion 2 to the second stock portion 3 to supply successively to outside from the micro-plate 10 at the uppermost stage and operation of containing the micro-plates for successively containing the micro-plates 10 delivered to receive from outside of the first stock portion 2 can be carried out by the same apparatus.

Thereby, in the case of repeating a plurality of times of operation constituting the object by the same micro-plate, when the micro-plate supplied from one side of the two micro-plates supplying and containing apparatus is contained to other side of the micro-plate supplying and containing apparatus, an order of stacking the micro-plates can always be maintained constant. Therefore, restacking operation which has been needed in the background art apparatus, that is, operation of switching to align an order of stacking the micro-plates in the respective stackers is dispensed with and an efficiency of test operation can be promoted.

Further, according to the micro-plate supplying and containing apparatus of the invention, when the micro-plates are taken out from the stacked member of the micro-plates sheet by sheet, the micro-plate at the uppermost stage is separated to take out and therefore, even in the case of constituting the object by the micro-plate in a state of being mounted with a lid at an upper face thereof, the micro-plate can be supplied stably by separating the mask plate.

### INDUSTRIAL APPLICABILITY

The micro-plate supplying and containing apparatus of the invention achieves an effect of capable of promoting an efficiency of test operation without needing restacking operation of the micro-plate and is useful for a use of automatically carrying out a test constituting an object by a plurality of mircoplates.

1. An apparatus of supplying and containing a micro-plate, comprising:
a first stock portion, including:
a first support member, supporting a plurality of micro-plates in a stacked state from a lower side; and
a support release mechanism, changing the first support member into a state of not supporting the micro-plates;
a second stock portion, including a long vertical length arranged on a lower side of the first stock portion in series in an up and down direction;
a second support member, supporting the plurality of micro-plates in the stacked state from a lower side at an inner portion of the second stock portion;
a moving up and down mechanism, moving up and down the second support member;
a micro-plate carrying unit, carrying the micro-plate disposed at a micro-plate carry out level set at the second stock portion to outside and mounting the micro-plate to the second support member from the outside;
a micro-plate supplying operation processing unit, making the support release mechanism and the moving up and down mechanism execute an operation of moving up the second support member and lifting the micro-plates in the stacked state supported by the first support member by the second support member to switch to mount on the second support member, thereafter, changing the first support member into a state of not supporting the micro-plates, thereafter, moving down the second support member and moving the plurality of micro-plates in the stacked state to the second stock portion, and disposing a micro-plate at a uppermost stage in the stacked state to the micro-plate carry out level by controlling a height position of the second support member; and
a micro-plate containing operation processing unit, making the moving up and down mechanism execute an operation of moving up the second support member mounted with the micro-plate delivered by the micro-plate carrying unit to a height of capable of supporting the micro-plate by the first support member and thereafter, supporting the micro-plate by the first support member by moving down the second support member.

2. The apparatus of supplying and containing a micro-plate according to claim 1, wherein the micro-plate carrying unit comprises:
a grasping mechanism for pressing a grasping member to a side face of the micro-plate at the uppermost stage disposed at the micro-plate carry out level to grasp;
a micro-plate separating unit, separating the micro-plate at the uppermost stage from the micro-plate at a successive stage by moving the second support member and the grasping member grasping the micro-plate relative to each other in the up and down direction; and
a micro-plate carrying out unit for carrying out the micro-plate at the uppermost stage separated by the micro-plate separating unit.

3. The apparatus of supplying and containing a micro-plate according to claim 1, further comprising:
an upper face detecting unit, detecting an upper face of the micro-plate at the uppermost stage in the plurality of micro-plates supported by the second support member in the stacked state;
wherein the micro-plate supplying operation processing unit disposes the micro-plate at the uppermost stage to the micro-plate carry out level by controlling the moving up and down mechanism by constituting a reference by the height position of the second support member when the upper face detecting unit detects the upper face of the micro-plate.

4. The apparatus of supplying and containing the micro-plate according to claim 3, wherein the upper face detecting unit is contact type detecting unit having a contactor brought into contact with the upper face of the micro-plate.

5. The apparatus of supplying and containing a micro-plate according to claim 4, wherein the contact type detecting unit includes:
a sensor, detecting that the contactor is moved to be brought into contact with the upper face of the micro-plate.

6. The apparatus of supplying and containing a micro-plate according to claim 2, wherein the micro-plate carrying out unit includes:
a support table, supporting the micro-plate at the uppermost stage by advancing to between the micro-plate at the uppermost stage and the micro-plate at the successive stage separated by the separating unit.

7. The apparatus of supplying and containing a micro-plate according to claim 2, wherein the micro-plate separating unit is a grasping member moving up and down mechanism for moving up and down the grasping member.

8. The apparatus of supplying and containing a micro-plate according to claim 3, further comprising:
a fixing mechanism, temporarily fixing the micro-plate at the successive stage when the micro-plate at the uppermost stage is separated.

9. The apparatus of supplying and containing a micro-plate according to claim 2, wherein the micro-plate carrying out unit is provided with the grasping mechanism.

10. The apparatus of supplying and containing a micro-plate according to claim 9, wherein the micro-plate separating unit is a grasping portion moving up and down mechanism for moving up and down the grasping mechanism and the grasping member moving up and down mechanism is provided to the micro-plate carrying unit.

11. The apparatus of supplying and containing a micro-plate according to claim 10, further comprising:
a fixing mechanism, temporarily fixing the micro-plate at the successive stage when the micro-plate at the uppermost stage is separated.

12. A method of supplying a micro-plate characterized in a method of supplying a micro-plate in an apparatus of supplying and containing a micro-plate comprising a first stock portion including a first support member for supporting a plurality of micro-plates in a stacked state from a lower side and a support release mechanism for changing the first support member into a state of not supporting the micro-plates, a second stock portion having a long vertical length arranged on a lower side of the first stock portion in series in an up and down direction, a second support member for supporting the plurality of micro-plates in the stacked state from a lower side at an inner portion of the second stock portion, a moving up and down mechanism for moving up and down mechanism for moving up and down the second support member, and micro-plate carrying unit for carrying the micro-plate disposed at a micro-plate carry out level set at the second stock portion to outside and mounting the micro-plate to the second support member from the outside, said method comprising the steps of:
moving up the second support member and lifting the micro-plates in the stacked state supported by the first support member by the second support member to switch to mount on the second support member;
changing the first support member into a state of not supporting the micro-plates by operating the support release mechanism;
moving down the second support member and moving the micro-plates in the stacked state which have been switched to mount on the second support member to the second stock portion;
disposing the micro-plate at a uppermost stage in the stacked state to the micro-plate carry out level by controlling a height position of the second support member; and
carrying out the micro-plate disposed at the micro-plate carry out level to the outside by the micro-plate carrying unit.

13. The method of supplying a micro-plate according to claim 12, further comprising the steps of:
detecting an upper face of the micro-plate at the uppermost stage by upper face detecting unit after switching the plurality of micro-plates in the stacked state to mount on the second support member;
wherein the micro-plate at the uppermost stage is disposed to the micro-plate carry out level by controlling the moving up and down mechanism by constituting a reference by a height position of the second support member when the upper face of the micro-plate is detected by the upper face detecting unit.

14. A method of containing a micro-plate characterized in a method of containing a micro-plate in an apparatus of supplying and containing a micro-plate comprising a first stock portion including a first support member for supporting a plurality of micro-plates in a stacked state from a lower side and a support release mechanism for changing the first support member into a state of not supporting the micro-plates, a second stock portion having a long vertical length arranged on a lower side of the first stock portion in series in an up and down direction, a second support member for supporting the plurality of micro-plates in the stacked state from a lower side at an inner portion of the second stock portion, a moving up and down mechanism for moving up and down the second support member, and micro-plate carrying unit for carrying the micro-plate disposed at a micro-plate carry out level of the second stock portion to outside and mounting the micro-plate to the second support member from the outside, said method comprising the steps of:
mounting the micro-plate to the second support member by the micro-plate carrying unit; and
moving down the second support member mounted with the micro-plate delivered by the micro-plate carrying unit to a height of capable of supporting the micro-plate by the first support member and thereafter supporting the micro-plate by the first support member by moving down the second support member.

15. An apparatus of processing a micro-plate characterized in comprising a first apparatus of supplying and containing a micro-plate capable of containing a plurality of micro-plates in a stacked state, a second apparatus of supplying and containing a micro-plate capable of containing a plurality of micro-plate in a stacked state, a working unit for executing a predetermined processing operation for the micro-plates, and a micro-plate carrying mechanism for carrying the micro-plates from the first apparatus of supplying and containing the micro-plate to the second apparatus of containing and supplying the micro-plate via the working unit, or carrying the micro-plates from the second apparatus of supplying and containing the micro-plate to the first apparatus of supplying and containing the micro-plate via the working unit;
wherein the first apparatus of supplying and containing the micro-plate and the second apparatus of supplying and containing the micro-plate each comprises:
a first stock portion, including:
a first support member for supporting a plurality of micro-plates in a stacked state from a lower side; and
a support release mechanism for changing the first support member into a state of not supporting the micro-plates;
a second stock portion, having a long vertical length arranged on a lower side of the first stock portion in series in an up and down direction;
a second support member, supporting the plurality of micro-plates in the stacked state from a lower side at an inner portion of the second stock portion;
a moving up and down mechanism, moving up and down the second support member;
a micro-plate carrying unit, carrying the micro-plate disposed at a micro-plate carry out level set at the second stock portion to outside and mounting the micro-plate to the second support member from the outside;
a micro-plate supplying operation processing unit, making the support release mechanism and the moving up and down mechanism execute an operation of moving up the second support member and lifting the micro-plates in the stacked state supported by the first support member by the second support member to switch to mount on the second support member, thereafter, changing the first support member into a state of not supporting the micro-plates, thereafter, moving down the second support member and moving the plurality of micro-plates in the stacked state to the second stock portion, and disposing the micro-plate at a uppermost stage in the stacked state to the micropalte carry out level by controlling a height position of the second support member; and
a micro-plate containing operation processing unit, making the moving up and down mechanism execute an operation of moving up the second support member mounted with the micro-plate delivered by the micro-plate carrying unit to a height of capable of supporting the micro-plate by the first support member and thereafter, supporting the micro-plate by the first support member by moving down the second support member.
